(19) United States
(12) Patent Application Publication
(10) Pub. No.: US 2024/0153619 A1
(43) Pub. Date: May 9, 2024

(54) A SYSTEM FOR SEAMLESS DISCHARGE OF PATIENTS FROM HOSPITALS

(71) Applicant: Vijayalakshmi Natarajan, Cupertino, CA (US)

(72) Inventor: Vijayalakshmi Natarajan, Cupertino, CA (US)

(21) Appl. No.: 18/385,880

(22) Filed: Oct. 31, 2023

Related U.S. Application Data
(60) Provisional application No. 63/422,550, filed on Nov. 4, 2022.

(51) Int. Cl. G16H 40/20

(52) U.S. Cl. CPC G16H 40/20

(57) ABSTRACT

Embodiments of the present disclosure provide a system for seamless discharge of patients from hospitals. A centralized electronically interconnected cloud-based system is described. The system provides Intelligent Patient Review which enables an Artificial Intelligence (AI) based analysis of patient profile inter relation between Diagnosis, Medication and Labs to alert diagnosis and medication errors. The system further provides a mechanism for hospitals to push patients' profiles from Electronic Health Record (EHR) systems and recommend the relevant care providers and broadcast the patient discharge readiness.

### CROSS REFERENCE OF RELATED APPLICATIONS

This application claims the benefits of U.S. provisional application No. 63/422,550 filed Nov. 4, 2022 and entitled “A SYSTEM FOR SEAMLESS DISCHARGE OF PATIENTS FROM HOSPITALS”, which provisional application is incorporated by reference herein in its entirety.

### TECHNICAL FIELD

The present disclosure relates to the hospital system for discharge of a patient. More particularly, it relates to a system for seamless, effective, efficient, integrated, and patient centric discharging of patients from hospitals that reduces potential risk for patients and providers during the transition of care across the post-acute continuum of care through a HIPAA compliant secure cloud based interoperable platform.

### BACKGROUND

Patients discharged from hospitals may need post-acute care. It triggers a post-acute transition and continuum of care process. The care process includes home health care, long term care, ambulatory care, pharmacy, durable medical equipment, ancillary medical and non-medical community providers, meal services, community housing, etc. The long-term care facilities include nursing homes, skilled nursing facilities, assisted living facilities, memory care facilities, independent living facilities, small home nursing homes and subacute.

The post-acute care providers and discharge planners/case managers in the acute would integrate, coordinate, and collaborate to provide post-acute services that patients need through the continuum of care recommended by healthcare providers (such as doctors) and work to finalize the discharge to the facilities.

For smooth discharge, the hospitals should provide complete guidance to the patients and families who need post-acute care. In addition, the hospitals should provide all the required information of the patients to the post-acute care providers. It is critical for the hospitals to ensure that accurate post-discharge instructions are shared with all the post-acute care providers through the continuum of care recommended by doctors and work to finalize the discharge to the facilities.

Nowadays, the hospitals go through a manual process to connect with post-acute care providers and exchange paperwork. For example, the hospital case managers may connect to the post-acute care providers by making phone calls and exchanging fax, eFax, or mails. In some cases, the list of discharging patients is shared as an online list. The process reduces the efficiency of discharge and increases the cost for the hospitals if the discharge takes time. The discharge process does not include a verification system to reconcile medications prescribed for the admission diagnosis, and for all post-acute follow up care. The current system is manual verification with the patient or responsible person for the patient.

Thus, there is a need for a system with seamless discharging of patients, which addresses the previously mentioned problems.

### SUMMARY

Consequently, there is a need for an improved method and arrangement for implementing a system that alleviates at least some of the above-cited problems.

It is therefore an object of the present disclosure to provide a system for seamless discharge of patients from hospitals to mitigate, alleviate, or eliminate all or at least some of the above-discussed drawbacks of presently known solutions.

This and other objects are achieved using a system and a method as defined in the appended claims. The term exemplary is in the present context to be understood as serving as an instance, example or illustration.

According to the first aspect of the present disclosure, a system for seamless discharge of patients from hospitals is disclosed. A centralized electronically interconnected hipaa compliant cloud-based system is described. The system provides Intelligent Patient Review which enables an Artificial Intelligence (AI) based analysis of patient profile inter relation between Diagnosis, Medication and Labs to alert diagnosis and medication errors.

The system further provides a mechanism for hospitals and different post-acute providers to push patients' profiles from Electronic Health Record (EHR) systems/Electronic Medical Records (EMR) and recommend the relevant/subsequent care providers and broadcast the patient discharge readiness.

The system further enables care providers to showcase the services and review the patients getting discharged and intake acceptable patients.

In addition, the system enables patients to go through the recommended and accepted providers. The patients can finalize the care provider of choice and connect with the care providers to set up appointments.

Such interactions happen in the central system which connects the above entities electronically. The system described in the present invention provides several benefits to hospitals, all the care providers, and patients. For example, hospitals benefit from centralized connected and faster discharge reducing cost and improving patient safety by avoiding medical/non-medical errors due to manual interactions. Care providers benefit from faster admission and avoid medical errors due to patient diagnosis/labs/medication analysis readily available through Intelligent.

Patient Review. Patients benefit from the ability to select the care of choice and faster and relevant start of continuum care.

In some embodiments, any of the above aspects may additionally have features identical with or corresponding to any of the various features as explained above for any of the other aspects.

### BRIEF DESCRIPTION OF THE DRAWINGS

The foregoing will be apparent from the following more particular description of the example embodiments, as illustrated in the accompanying drawings in which reference characters refer to the same parts throughout the different views. The drawings are not necessarily to scale, emphasis instead being placed upon illustrating the example embodiments.

FIG. 1 illustrates a block diagram of a system for seamless discharge of patients from hospitals, according to some embodiments of the present invention.

FIG. 2 illustrates a flow diagram of a patient module of Integrated Continuum Care, according to some embodiments of the present invention.

FIG. 3 illustrates a flow diagram of a hospital module of Integrated Continuum Care, according to some embodiments of the present invention.

FIG. 4 illustrates a flow diagram of a care provider module of Integrated Continuum Care, according to some embodiments of the present invention.

FIG. 5 illustrates a flow diagram of an intelligent patient review module of Integrated Continuum Care, according to some embodiments of the present invention.

FIG. 6 illustrates a schematic block diagram illustrating an example apparatus, according to some embodiments of the present invention; and

FIG. 7 illustrates an example computing environment implementing a system for seamless discharge of patients from hospitals, according to some embodiment of the present invention.

### DETAILED DESCRIPTION

Aspects of the present disclosure will be described more fully hereinafter with reference to the accompanying drawings. The apparatus and methods disclosed herein can, however, be realized in many different forms and should not be construed as being limited to the aspects set forth herein. Like numbers in the drawings refer to like elements throughout.

Reference will now be made in detail to embodiments, examples of which are illustrated in the accompanying drawings. In the following detailed description, numerous specific details are set forth to provide a thorough understanding of the present invention. However, it will be apparent to one of ordinary skills in the art that the present invention may be practiced without these specific details. In other instances, well-known methods, procedures, components, circuits, and networks have not been described in detail so as not to unnecessarily obscure aspects of the embodiments.

The terminology used herein is for the purpose of describing particular aspects of the disclosure only and is not intended to limit the invention. It should be emphasized that the term “comprises/comprising” when used in this specification is taken to specify the presence of stated features, integers, steps, or components, but does not preclude the presence or addition of one or more other features, integers, steps, components, or groups thereof. As used herein, the singular forms “a”, “an”, and “the” are intended to include the plural forms as well, unless the context clearly indicates otherwise.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Embodiments of the present disclosure will be described and exemplified more fully hereinafter with reference to the accompanying drawings. The solutions disclosed herein can, however, be realized in many different forms and should not be construed as being limited to the embodiments set forth herein.

It will be appreciated that when the present disclosure is described in terms of a method, it may also be embodied in one or more processors and one or more memories coupled to the one or more processors, wherein the one or more memories store one or more programs that perform the steps, services, and functions disclosed herein when executed by the one or more processors.

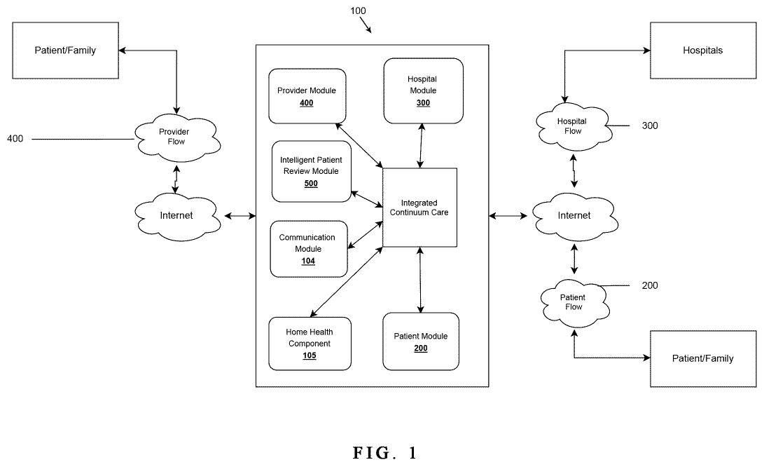

FIG. 1 illustrates a block diagram of a system 100 for seamless discharge of patients from hospitals, according to some embodiments of the present invention. The system 100 may comprise a plurality of modules for seamless discharge of patients from the hospitals. For example, the system 100 may comprise a patient module 200, a hospital module 300, a care provider module 400, and an intelligent patient review module 500. Each module is communicatively coupled with an integrated continuum care (ICC) unit for exchanging information related to the patient profile with each other.

The ICC is a centralized cloud based HIPPA compliant system which helps to electronically connect and transact between hospitals, care providers (all post acute providers, such as Skilled Nursing Facility (SNF) and home health, ancillary providers like pharmacy, durable medical equipment, transportation and all medical and non-medical providers and entities) and patients. The system 100 provides end to end service from patient's hospital discharge to the availing continuum of care. Hospitals benefit from faster discharge reducing cost and importantly ensuring patient safety when transitioning across the post-acute continuum of care and patients benefit from faster start and appropriate and appropriate and necessary of post-acute continuum of care. The modules enable the seamless execution of this process by providing different services as described henceforth. Hospitals may create a profile detailing their services and create logins for all case managers and any person who is responsible for patient discharge. When a patient is ready to be discharged, case managers may invite ready to be discharged patients to create profiles in the system. The invited patients may create login/profile in the system 100. The profile may include name, insurance, demographic profile, living situation, assistance required, etc.

Further, care providers like Post-Acute Care, home health may create registration for each organization or facility, update profile, include photos, videos of the facility, map, live tour option, 3D map/tour of the facility, text chat/video chat with healthcare providers (clinical and non-clinical). Furthermore, Hospital personnel (case workers, discharge planners, social workers, transitional care nurses) are able to search for post-acute care providers, based on preferred geographical location, insurance covered, name of provider, license type, availability of service, star rating from CMS/testimonials from patients/families.

Once a short list of care providers is identified by hospital personnel then the care providers are notified of the patient discharge. The relevant clinical documentation, labs, medications, transportation, and discharge disposition (living situation, meals, follow up appointments in the community) is updated during the discharge process. follow up labs, appointments are updated at the time of discharge.

The hospital may provide the patient profile to shortlisted care providers. When the patient is being reviewed by the skilled nursing facility or nursing home, the SNF clinical and non-clinical staff are able to quickly review the clinical notes capturing key words, through AI features regarding diagnosis, H&P, medications and general functional level of the patients to ensure the patient is a good fit for their facility. The SNF or nursing facility can estimate the cost of the admission, labor hours required to care for the patient. Health care providers are able to accept or deny the service based on the scope of care needed, availability of staff, insurance or cost coverage etc.

The hospital may provide details of the care providers to the patients or families. The patient may accept or deny the care providers based on the review and have an option to choose their preferred care provider. Once a care provider is selected by patient or family, the care providers (SNF/home care) are notified and relevant up to date clinical documentation, labs, medications and discharge disposition is electronically sent through the interoperable EMR/EHR during the discharge process. The hospitals may update follow up labs and appointments at the time of discharge. Patients' demographic and health care information will flow from one level of care into the next electronically without having to repeatedly enter the same demographic, financial and clinical diagnosis medications etc. Labs in hospital, SNF, Home health, Assisted Living Facility (ALF) and outpatient will be connected to ensure seamless connection of records. Families are able to access the portal during the care transition. Acute and post-acute care providers across the continuum of care have the option of having their own site specific EHR/EMR updated with the discharge coordination process that is transacted through the interoperable centralized communication (system 100).

The system 100 will extrapolate clinical info regarding durable medical equipment (DME), transportation needs, Home health or other post-acute clinical needs such as but not limited to residential arrangements, meals after discharge, verify insurance coverage in the background, contact the respective providers and send notifications and enable 2-way communication and confirm schedule of follow up and delivery.

SNF admission portal may message the patient's family, patients assigned physician, assigned staff that patient is admitted with room number, name, and basic clinical diagnosis and physician name to everyone involved in care of the patient (dietary, rehab, business office, and social service). Family communication component (ability to schedule visitation appointment, file grievance, complaint, updating personal belongings inventory, updating insurance details, diet preferences, teleconferencing option), access EHR/EMR upon request, discharge planning to next step down level of care.

Further, discharge planning components (3D video tour, maps, photos of the facility and team members, patient room, pay copay for insurance etc.) may be provided. Home Health component may be able to schedule time of initial visit, with name of visiting healthcare professional, gender, languages spoken, services needed (clinical or non-clinical), equipment needed, message family, attending physician from home health and primary physician about follow care and update for progress, changes in condition and transfer to hospital or ER or ALF or SNF), able to teleconference. Staff scheduling calendar portal along with time of preferred visit to home is also available on the platform.

Once discharge to a post-acute provider is confirmed, the post-acute provider and family or patient may be able to schedule times of visits, sharing progress as preferred. All information that is needed for patient care from acute to post-acute setting during transitional care is coordinated in one place and followed up on. Subsequent transfers from one post-acute setting to another are also tracked and communicated to all the post-acute care providers. In addition, patients and family are able to review each level of care and rate and submit in the system 100.

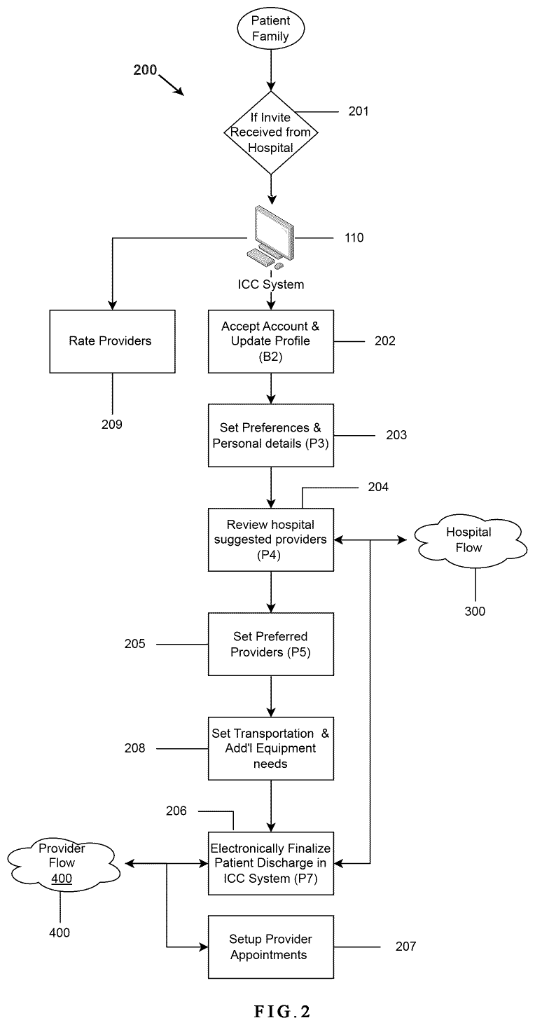

The patient module 200 may electronically interact (approve, sign documents, pay) with hospitals and care providers to enable the patients to select acceptable care providers and may help in connecting with the right care providers of continuum care. The patient module 200 may be explained in detail with reference to FIG. 2.

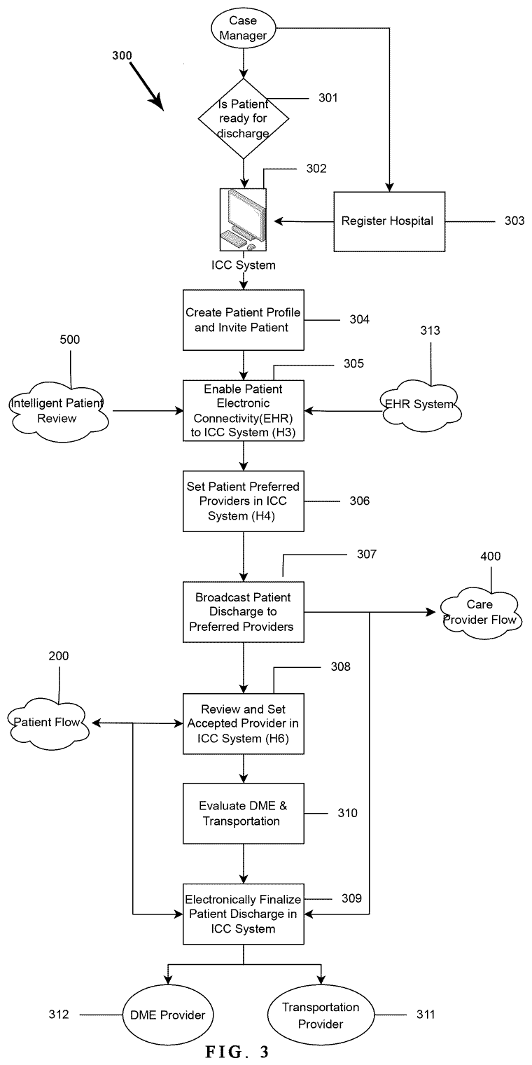

The hospital module 300 may electronically interact with patients and care providers to enable patients onboarding using EHR/EMR connectivity for sharing of patient diagnosis
and electronic contract management. The hospital module 300 may be explained in detail with reference to FIG. 3.

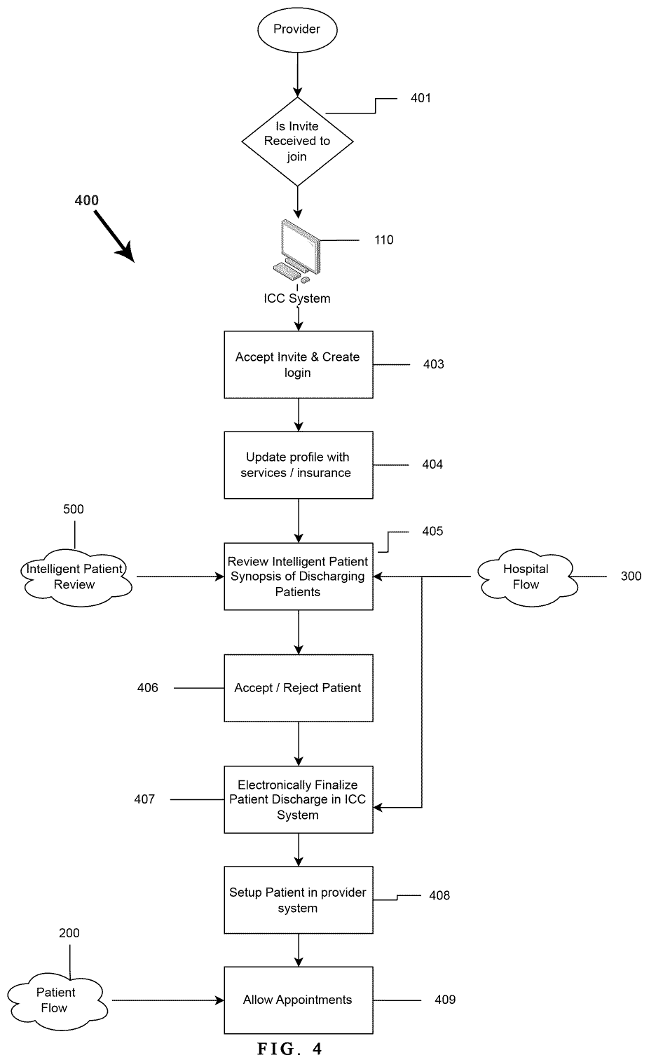

The care provider module 400 may electronically interact with hospitals and patients to enable intelligent patient diagnosis review, patient acceptance, contract management. The care provider module 400 may be explained in detail with reference to FIG. 4.

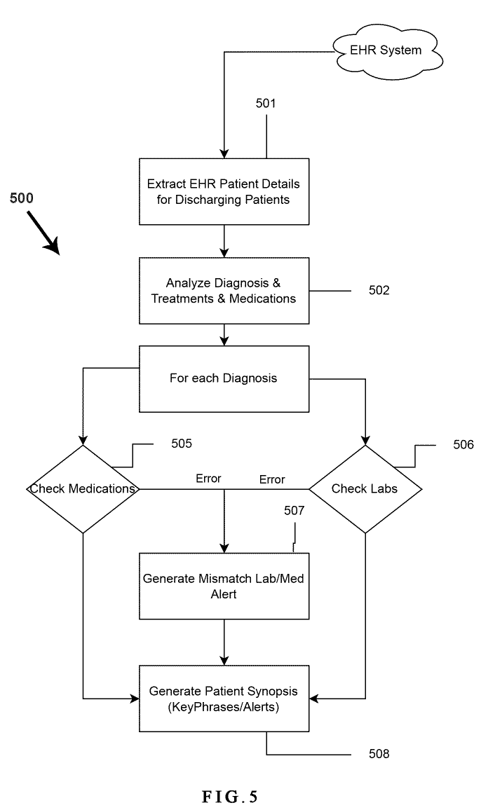

The intelligent patient review module 500 may utilize Artificial intelligence (AI) to review Patient diagnosis, labs, and medication to come up with a Patient Synopsis or Alerts and may raise any irrelevant treatments and errors for care providers to come up with a continuum care plan. The intelligent patient review module 500 may be explained in detail with reference to FIG. 5.

Each module may be communicatively coupled with the ICC through a communication module 104. The communication module 104 may enable EHR/EMR connected Patient's data extraction and push to provider systems. The details extracted and pushed includes lab tests with upcoming lab work, pharmacy details with ongoing prescriptions, transportation needs and respective providers. Once a care provider is selected by patient and patient or family consent to transfer. The care providers (SNF/home care) may be notified and relevant clinical documentation, labs, medications and discharge disposition is electronically sent in the discharge process. follow up labs, appointments are updated at the time of discharge.

Patients' demographic and health care information may be transmitted from one level of care into the next electronically without having to repeatedly enter the same demographic, financial and clinical diagnosis medications etc. Labs in hospital, SNF, Homehealth, ALF and outpatient will be connected to ensure seamless connection of records. Families are able to access the portal during the care transition.

At time of discharge, the system will extrapolate clinical info regarding providing durable medical equipment (DME), transportation needs (either or both together) and verify insurance coverage in the background, contact the respective providers and send notifications and enable two-way communication and confirm schedule of follow up, delivery.

Further, the communication module 104 has ability to schedule visitation appointment, file grievance, complaint, updating personal belongings inventory, updating insurance details, diet preferences, teleconferencing option), access EHR/MR upon request, discharge planning to next step-down level of care discharge planning component (3D video tour, maps, photos of the facility and team members, patient room, pay and copay for insurance etc).

The ICC further comprise a home health component 105 for scheduling time to initial visit with name of visiting healthcare professional, gender, languages spoken, services needed (clinical/non-clinical), equipment needed, message family, attending physician from home health and primary physician about follow care and update for progress, changes in condition and transfer to hospital/ER/ALF/SNF), able to teleconference. Staff scheduling calendar portal along with time of preferred visit to home is also an available platform.

The ICC may provide a central care coordination. All information that is needed for patient care from acute to post-acute setting during transitional care is coordinated in one place and followed up on. Subsequent transfers from one post-acute setting to another are also tracked and communicated to all the post-acute care providers.

FIG. 2 illustrates a block diagram of the patient module 200, in accordance with an embodiment of the present invention. After treatment of patients, the hospital needs to discharge the patients. The journey of the discharging process starts with the registration process in the ICC. Firstly, the hospital sends an email inviting to the patients to be discharged, at step 201.

At step 202, The patient may accept the invite and update his profile. As the patient is ready to be discharged from the hospital, the Hospital Module invites the patient to the system. The patient uses the invite in email to create a login and profile in system.

At step 203, the patients may update their personal details with preferences in continuum care. At step 208, the patients may review the care providers suggested and accepted by the hospital. The hospital may set recommended care providers when discharging patients and broadcast them to the care providers through the system. Providers get to review the synopsis created by the Intelligent Patient Review module and accept patients.

At step 205, The patients may set a preferred provider to get discharged in the system after review. At step 206, the patients may accept the contract electronically from the hospital & care providers to start the continuum care. The hospitals may also set contracts with the equipment providers as needed. Further, at step 207, the patient may start appointments with the preferred provider.

FIG. 3 illustrates a block diagram of the hospital module 300, in accordance with an embodiment of the present invention. At step 301, a patient is ready to discharge. At step 302, a case manager assigned for the patient goes to the ICC system on the web.

At step 303, hospitals register hospital profiles with an account for each case manager responsible for discharge planning. Hospitals may create a profile detailing their services and create logins for all case managers and any person who is responsible for patient discharge.

At step 304, each Case manager when starting to plan for the Patient Discharge may create a patient profile for discharging patients. When a patient is ready to be discharged, case managers invite ready to be discharged patients to create profiles in the system. Invited patients (Patient Module) create login/profile in the system.

The Hospital may update profiles including name, insurance and demographic, living situation, assistance required (like wheelchair), bed requirement like male/female bed, bed location like near window/near door. These details help providers in accepting the right patient based on what is offered in their facility.

At step 305, the hospital may enable EHR/EMR Access for the system to get discharge details for the discharging patients like diagnosis, labs, medications and related details. Costs of medication are calculated based on the patient insurance by the platform. Feasibility of referral, risk factor and discharge potential are updated by the hospital in the patient profile.

The hospital may provide discharge scheduling to the care providers (home health & skilled nursing, DME, transportation etc.). The Intelligent Patient review module may analyze and recommend a list of care providers based on the EHR provided diagnosis/labs/medications and other details.

At step 306, hospital personnel (case workers, discharge planners, social workers, or transitional care nurses) may be able to search for post-acute care providers, based on preferred geographical location, insurance covered, name of provider, license type, availability of service, star rating from CMS/testimonials from patients/families and set preferred providers.

At step 307, the hospital may broadcast the patient discharge information to the set of preferred providers selected in step 306. At step 308, once a set of providers accept the patient, the hospital may review and communicate to the patient. The patient sets the preferred provider from the list communicated by the hospital. At step 309, the hospital may enable an electronic contract for the provider to start the continuum care.

FIG. 4 illustrates a flow diagram of a care provider module 400 of the ICC, according to some embodiments of the present invention. At step 403, the care providers like post-acute care, home health may create registration for each organization or facility, update profile, include photos, videos of the facility, map, live tour option, 3D map or tour of the facility, text chat or video chat with healthcare providers (clinical or non clinical). Further, the care provider may update available beds by category like window or door as it changes. The data may be pushed to the central platform.

At step 405, the hospital may broadcast the discharging patients to the relevant providers. Intelligent Patient Module may create a synopsis of patients for the care provider's review. Further, the hospital may provide feasibility of referral, risk factor and discharge potential for discharging patients to enable accepting the right patients to service. Furthermore, the hospital may provide transportation needs and labs applicable to both types of providers to connect with. In addition, skilled nursing providers get to review bed requirements like window or door beds.

At step 406, The care provider may accept or reject the request from the patients by looking at the synopsis or profile update from hospitals including bed availability, risk factor, cost of care involved and other relevant details. The SNF or nursing facility can estimate the cost of the admission, labor hours required to care for the patient. Health care providers are able to accept or deny based on the scope of care needed, availability of staff, insurance or cost coverage etc.

At step 407, the care providers may electronically accept or reject contracts from hospitals and patients. At step 408, the care provider may set up a patient in the provider system. At step 409, the care provider may allow appointments with the patients. Details of skilled nursing updates, staffing availability and assigned nursing staff are provided to accepted patients. Further, a co-pay portal may be registered for patients if needed by the care providers.

FIG. 5 illustrates a flow diagram of an intelligent patient review module 500 of the ICC, according to some embodiments of the present invention. At step 501, the intelligent patient review module 500 may extract medications and labs information of discharged patients. At step 502, the intelligent patient review module 500 may analyze diagnosis, treatments, and medications of the discharged patients. At step 505, the intelligent patient review module 500 may check medications of the discharged patients. At step 506, the intelligent patient review module 500 may check labs associated with the discharged patients. Further, at step 507, the intelligent patient review module 500 may come up with mismatched medication or lab reports.

At step 508, the intelligent patient review module 500 may generate patient synopsis. When the patient is being reviewed by the skilled nursing facility or nursing home, the SNF clinical and non-clinical staff are able to quickly review the patient synopsis capturing key words, through AI features regarding diagnosis, H&P, medications and general functional level of the patients to ensure the patient is a good fit for their facility cost.

This invention enables the faster discharge of patients to the approved provider thru direct connection electronically in addition with secured exchange of information. It uses the secured Electronic Medical Record (EMR/EHR) connectivity between the facilities to enable the faster discharge of patients and increase the productivity of the hospital care providers and most importantly ensure increased patient safety during transition across the continuum of care.

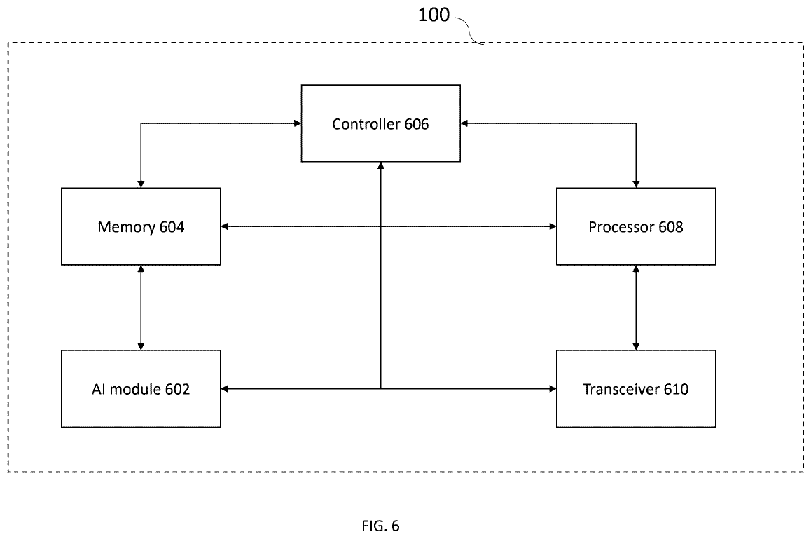

FIG. 6 illustrates a schematic block diagram illustrating an example apparatus, according to some embodiments of the present invention. The system 100 may be capable of electronically connecting and transacting between hospitals, care providers (such as Skilled Nursing Facility (SNF) and home health and all other medical and non-medical providers and entities) and patients and provides an option to have the coordination process that is coordinated and collaborated between providers documented in their site specific EHR/EMR is they chose to have that documentation transacted from system 100.

According to at least some embodiments of the present invention, the system 100 in FIG. 6 comprises one or more modules. These modules may e.g. be an artificial intelligence (AI) module 602, a memory 604, a controller 606, a processor 608, and a transceiver 610. The controller 606, may in some embodiments be adapted to control the above-mentioned modules.

The AI module 602, the memory 604, the processor 608, and the transceiver 610 as well as the controller 606, may be operatively connected to each other. Optionally, the transceiver 610 may be adapted to receive an instruction for usage of the system 100.

The controller 606 may be adapted to control the steps as executed by the system 100 according to the instructions received by the transceiver 610. For example, the controller 606 may be adapted to activate the AI module 602 (as described above in conjunction with the FIG. 1). Further, the processor 608 is adapted to perform the method and FIG. 1 in conjunction with the controller 606.

The memory 604 is adapted to store the patient information and the AI module 602 is adapted to process the stored information to find a suitable care provider for the patient.

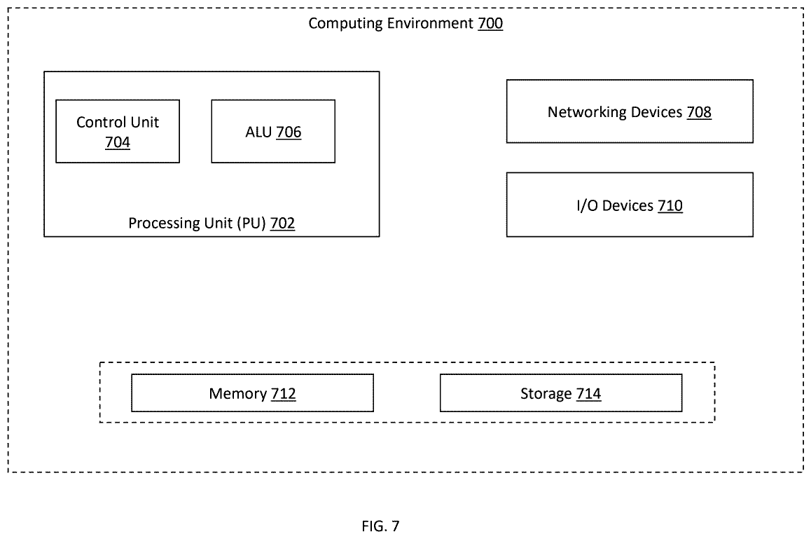

FIG. 7 illustrates an example computing environment 700 implementing a system for electronically connecting and transacting between hospitals, care providers (such as Senior Nursing Facility (SNF) and home care) and patients, according to some embodiment of the present invention. As depicted in FIG. 7, the computing environment 700 comprises at least one processing unit 702 that is equipped with a control unit 704 and an Arithmetic Logic Unit (ALU) 706, a plurality of networking devices 708 and a plurality Input output, I/O devices 710, a memory 712, and a storage 714. The processing unit 702 may be responsible for implementing the method described in FIGS. 1-5. For example, the processing unit 702 may in some embodiments be equivalent to the processor of the network node described above in conjunction with the FIGS. 1-5. The processing unit 702 is capable of executing software instructions stored in memory 712. The processing unit 702 receives commands from the control unit 704 in order to perform its processing. Further, any logical and arithmetic operations involved in the execution of the instructions are computed with the help of the ALU 706.

The computer program is loadable into the processing unit 702, which may, for example, be composed in an electronic apparatus. When loaded into the processing unit 702, the computer program may be stored in the memory 712 associated with or within the processing unit 702. According to some embodiments, the computer program may, when loaded into and run by the processing unit 702, cause execution of method steps according to, for example, any of the methods illustrated in FIGS. 1-5 or otherwise described herein.

The overall computing environment 700 may be composed of multiple homogeneous and/or heterogeneous cores, multiple CPUs of different kinds, special media and other accelerators. Further, the plurality of processing unit 702 may be located on a single chip or over multiple chips.

The algorithm consisting of instructions and codes required for the implementation are stored in either the memory 712 or the storage 712 or both. At the time of execution, the instructions may be fetched from the corresponding memory 712 and/or storage 712 and executed by the processing unit 702.

In case of any hardware implementations, various networking devices 708 or external I/O devices 710 may be connected to the computing environment to support the implementation through the networking devices 708 and the I/O devices 710.

The embodiments disclosed herein can be implemented through at least one software program running on at least one hardware device and performing network management functions to control the elements. The elements shown in FIG. 7 include blocks which can be at least one of a hardware device, or a combination of hardware device and software module.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the disclosure.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the disclosure.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user's computer or mobile device, wristband, smartphone, or any suitable combination thereof. Other systems and methods of the embodiment can be embodied and/or implemented at least in part as a machine configured to receive a computer-readable medium storing computer readable instructions. The instructions can be executed by computer-executable components integrated with apparatuses and networks of the type described above. The computer-readable medium can be stored on any suitable computer-readable media such as RAMs, ROMs, flash memory, EEPROMs, optical devices (CD or DVD), hard drives, and floppy drives, or any suitable device. The computer-executable component can be a processor but any suitable dedicated hardware device can (alternatively or additionally) execute the instructions.

As a person skilled in the art will recognize from the previous detailed description and the FIGS. and claims, modifications and changes can be made to the embodiments of the invention without departing from the spirit and scope of this invention as defined in the following claim.

What is claimed is:

1. A method for seamless discharge of patients from hospitals, the method comprising:
providing, by a patient, a profile to a server at a time of discharge from a hospital;
extracting, by a care provider, the profile of the patient for providing a service to the patient;
shortlisting, by a hospital, the care provider from a plurality of care provider suitable for providing appropriate service to the patient;
providing, by the hospital, information about the shortlisted care provider to the patient;
approving or denying, by the patient, the shortlisted care provider based on a requirement of the patient; and
executing, by the care provider, a required service suitable for the patient when the patient approves the shortlisted care provider.
